(19) United States
(12) Patent Application Publication
(10) Pub. No.: US 2012/0035500 A1
(43) Pub. Date: Feb. 9, 2012

(54) Flexible Endoscopic Ultrasound Guided Biopsy Device

(76) Inventor: Shawn Ryan, Upton, MA (US)

(21) Appl. No.: 13/020,479

(22) Filed: Feb. 3, 2011

Related U.S. Application Data
(60) Provisional application No. 61/301,664, filed on Feb. 5, 2010.

(51) Int. Cl. A61B 10/02

(52) U.S. Cl. 600/566; 600/567

(57) ABSTRACT

A needle for insertion into a living body along a tortuous path comprises an elongate body extending between a proximal end which, during use, remains external to the body and a distal end which, when in an operative configuration, is positioned adjacent to a target structure within the body, a distal portion of the needle including a lumen extending therethrough to a tissue receiving opening at the distal end, the lumen having an inner diameter of at least 0.035 inches and a bending stiffness no greater than 0.08 Nm².

### PRIORITY CLAIM

This application claims the priority to the U.S. Provisional Application Ser. No. 61/301,664, entitled “FLEXIBLE ENDOSCOPIC ULTRASOUND GUIDED BIOPSY DEVICE” filed Feb. 5, 2010. The specification of the above-identified application is incorporated herewith by reference.

### BACKGROUND

Needle biopsy procedures are common for the diagnosis and the staging of disease. In particular, in endoscopic ultrasound-guided fine needle aspiration (EUS-FNA), the needle is advanced under ultrasound guidance so that the physician is able to visualize a position of the needle in relation to the target tissue. Thus, EUS-FNA ensures that the correct tissue is sampled while minimizing risk to the patient. Although EUS-FNA is a highly sensitive and specific procedure, it is often difficult to acquire a suitable sample under certain clinical situations. For example, needles designed for procedures requiring navigation along tortuous paths to target sites from which it is desired to collect tissue, are generally small in diameter and formed of flexible materials. However, in certain situations it may desirable to use a needle with a larger inner diameter even though the needle must be advanced along a tortuous path. Such a larger inner diameter needle must still have an axial compressive stiffness sufficient to penetrate tissue, including even tougher tissue (e.g., fibrotic tissue masses), while maintaining the flexibility required to navigate to the target site along a tortuous path.

The most common current EUS-FNA needle device is a 22 ga stainless steel needle having an outer diameter of 0.028″ and an inner diameter of approximately 0.020″ with an elastic modulus of 28,000,000 psi. Although larger diameter needles are available, the bending stiffness of these devices is significantly greater than for the smaller diameter needles making it difficult to navigate these larger diameter needles along the tortuous paths through which the 22 ga needles may be passed. The increased wall thickness of these larger diameter needles is necessary to reduce the risk of structural failure due to small defects in the material or as a result of compressive stresses on the needle as the needle is moved along a curved path.

### SUMMARY OF THE INVENTION

The present invention is directed to a needle for insertion into a living body along a tortuous path comprising an elongate body extending between a proximal end which, during use, remains external to the body and a distal end which, when in an operative configuration, is positioned adjacent to a target structure within the body, a distal portion of the needle including a lumen extending therethrough to a tissue receiving opening at the distal end, the lumen having an inner diameter of at least 0.035 inches and a bending stiffness no greater than 0.08 Nm²

### BRIEF DESCRIPTION OF THE DRAWINGS

FIG. 1 shows a side view a distal portion of a device according to an exemplary embodiment of the present invention; and

FIG. 2 shows a cross-sectional view of the device of FIG. 1, along line A-A.

### DETAILED DESCRIPTION

The present invention may be further understood with reference to the following description and the appended drawings, wherein like elements are referred to with the same reference numerals. The present invention relates to endoscopic devices and, in particular, relates to an improved endoscopic device having a larger inner diameter while maintaining a flexibility sufficient to permit navigation of the device along a tortuous path. For example, the most common EUS-FNA device is a 22 ga needle having a lumen with an inner diameter of approximately 0.020″. The 22 ga needle is most commonly used because of its low bending stiffness, which allows the 22 ga needle to traverse the winding paths of body lumens. Although, in certain diagnostic and/or therapeutic applications, a lumen of at least 0.035″ (corresponding to a 19 ga needle) would be preferably, such needles are not utilized because the needles having such larger inner diameters generally show a bending stiffness that prevents the needle from traversing the winding paths of natural body lumens in the manner of the 22 ga needles.

Exemplary embodiments of the present invention describe an endoscopic biopsy device formed of a material permitting the device to include a lumen having an inner diameter of at least 0.035″ and a bending stiffness of no greater than 0.08 Nm². Conventional EUS-FNA materials and construction do not permit such a low bending stiffness value for needles including an inner diameter of at least 0.035″. The devices according to the invention are capable of passing through the body along tortuous paths (e.g., within natural body lumens) while maintaining a larger inner diameter for collecting tissue samples and/or transferring fluids.

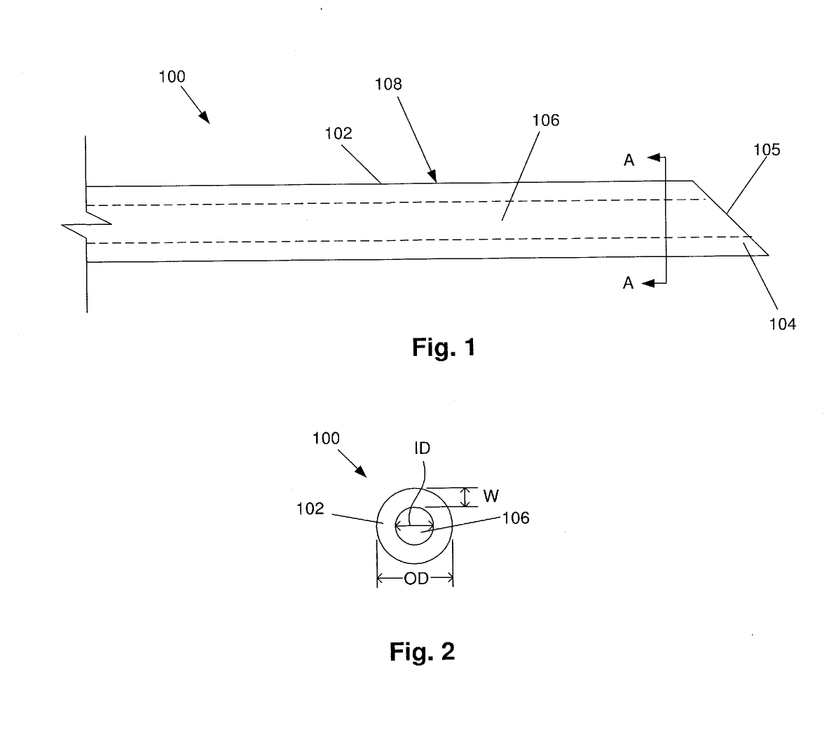

As shown in FIG. 1, a device 100 according to an exemplary embodiment of the present invention comprises a longitudinal element 102 extending from a proximal end (not shown) to a distal end 104 with a lumen 106 extending therethrough. The distal end 104 may include a tapered tip for piercing tissue masses or surfaces. A tissue sample may be collected within the lumen 106. In a further embodiment, a syringe or other medical device may be coupled to the proximal end to facilitate the transfer of fluids through the lumen 106 to/from a target area via the distal end 104. A distal portion 108 of the longitudinal element 102 is formed of a material that permits the lumen 106 to include an inner diameter (ID) of at least 0.035″ (e.g., an inner diameter of a typical 19 ga needle) while maintaining a bending stiffness less than or equal to 0.08 Nm² such that the device 100 is navigable through a tortuous path in a manner that approaches conventional 22 ga needles. A proximal portion of the device 100 extending from the distal portion 108 to a proximal end of the device 100 may be formed as a solid tube or coil of another material such as stainless steel to maintain the desired axial strength and bending stiffness of the device 100. Where the proximal portion is formed as a coil, the coil will be sealed with a flexible membrane or other lining to transmit fluid/suction along an entire length of the lumen 106. As will be understood by those skilled in the art, the lumen 106 extends to a tissue receiving opening 105 in the distal end 104 which may be formed via an angled cut of the distal end 104 of the needle 100 or as a lateral opening in a wall of the needle 100.

It will be understood by those of skill in the art that bending stiffness is a key factor in determining the degree of bending a device is suited for in traversing tortuous paths. The bending stiffness is determined by the product of an elastic modulus of the material, E, and an area moment of inertia, I. The elastic modulus E is defined as the ratio of stress to strain and, for many materials, shows a range through which E is substantially constant changing at a yield point to a non-linear relationship. Strains in this non-linear range (i.e., beyond the yield point) cause plastic deformation of the material. The area moment of inertia, I, may be calculated using the formula, I=(π/64)(OD⁴−ID⁴). Thus, it will be understood by those of skill in the art that bending stiffness may be reduced by reducing a wall thickness W (i.e., a difference between the outer diameter OD and the inner diameter ID). However, reducing the wall thickness may compromise the structural integrity of the device 100. For example, improperly reduced wall thickness may lead to structural failures resulting from small defects in the device 100 and/or compressive stresses (e.g., “kinks”) when the device 100 is passed along a curved path. Thus, the distal portion 108 will include an outer diameter OD that does not exceed 0.052″, and more preferably does not exceed 0.047″, such that the wall thickness W is between 0.001″ and 0.008″. It will be understood by those of skill in the art, however, that the wall thickness W will may depend on several factors including, for example, defect size in the raw material, path tortuosity, axial loading, etc. Nitinol shows a value of E which is lower so that, for a needle of the same cross-sectional area, the bending stiffness will be lower while the yield point is not reached until levels of stress greater than those to which the needles are to be subjected during normal use so that a 19 gauge Nitinol needle shows the required bending stiffness without the plastic deformation associated with needle failure.

In a preferred embodiment, the distal portion 108 of the device 100 is formed of a material having an elastic modulus E that ranges between 7,000,000 to 10,000,000 psi. In a preferred embodiment, the device 100 may be formed of Nitinol. However, materials such as copper, titanium and aluminum may also be used. As would be understood by those skilled in the art, copper or brass needles need to be coated with a biocompatible material for use within the body. Furthermore, the bending stiffness of Titanium is greater than that of Nitinol making it slightly less suitable for this application. A device 100 formed of Nitinol allows the inner diameter of the lumen to be made at least 0.035″ while maintaining a bending stiffness substantially lower, up to 75% lower, than currently used materials such as stainless steel. The Nitinol is able to reduce the bending stiffness, enhancing torquability, while also maintaining the preferred wall thickness. Nitinol also provides higher compressive stiffness than other materials such as plastics such that the compressive stiffness is sufficient to enable the distal end 104 to penetrate even fibrous lesions and maintains the lumen 106 in a sealed configuration such that fluid may be transferred through the lumen 106 to the target area.

Nitinol also possesses other unique mechanical properties, which may provide further benefit to the device 100. For example, by selecting a transition temperature below room temperature the device 100 will have superelastic properties. Thus, it will be understood by those of skill in the art that the device 100 may be passed along tortuous paths multiple times without permanent deformation. That is, after being passed along a tortuous path, any deformation of the device 100 may be reversed by returning the device 100 to its memorized shape. For example, after use, the temperature of the device 100 may be lowered below its critical temperature and then raised back above the critical temperature to return the device 100 to its original shape. The superelastic property along with the elastic modulus E of Nitinol also permit the transmission of torque from the proximal end of the device 100 to the distal end 104 thereof to generate usable rotation of the distal end 104 of the longitudinal element 102 via rotation of any other portion of the longitudinal element 102, even while the device 100 extends along a tortuous path. Alternatively, the superelasticity and ease of shape setting may enable improved cutting or biopsy features.

As described above, the distal portion 108 of the longitudinal element 102 is preferably formed of Nitinol. However, it will be understood by those of skill in the art that an entire length of the longitudinal element 102 may also be formed of nitinol. Where only the distal portion of the longitudinal element 102 is formed of nitinol, a remaining portion of the longitudinal element may be formed of an alternate material such as, for example, stainless steel. In a further embodiment, the remaining portion of the longitudinal element 102 may include slots and/or other features formed therein to reduce the bending stiffness of the device 102.

It will be apparent to those skilled in the art that variations can be made in the structure and methodology of the present invention, without departing from the spirit and the scope of the invention. Thus, it is intended that the present invention cover the modifications and variations of this invention provided that they come within the scope of the appended claims and their equivalents.

What is claimed is:

1. A needle for insertion into a living body along a tortuous path, comprising:
an elongate body extending between a proximal end which, during use, remains external to the body and a distal end which, when in an operative configuration, is positioned adjacent to a target structure within the body, a distal portion of the needle including a lumen extending therethrough to a tissue receiving opening at the distal end, the lumen having an inner diameter of at least 0.035 inches and a bending stiffness no greater than 0.08 Nm².

2. The needle of claim 1, wherein the distal portion is formed of Nitinol.

3. The needle of claim 1, wherein the distal end is formed of one of copper, aluminum and titanium.

4. The needle of claim 1, wherein a thickness of a wall of the distal portion is between 0.001″ and 0.008″.

5. The needle of claim 2, wherein a critical temperature of the Nitinol is selected to be less than a temperature in an operative environment for the needle and wherein a desired shape for the needle is memorized for temperatures above the critical temperature so that the desired shape of the needle may be restored after use.

6. The needle of claim 5, wherein the critical temperature is selected to be less than a body temperature of the body.

7. The needle of claim 1, wherein tissue receiving opening is formed laterally through a wall of the distal portion.

8. The needle of claim 1, wherein a proximal portion is formed as a coil coupled to the distal portion.

9. The needle of claim 8, wherein the coil is sealed to permit transmission of one of a fluid and suction along an entire length thereof.

10. The needle of claim 1, wherein a proximal portion is formed as a solid mass of a material different from a material of which the distal portion is formed with an outer diameter smaller than that of the distal portion.

11. The needle of claim 1, wherein a proximal portion is formed as a solid mass of a material different from a material of which the distal portion is formed, the proximal portion including cut-outs reducing a bending stiffness thereof

12. The needle of claim 1, wherein a proximal portion is formed of stainless steel.

13. The needle of claim 1, wherein the distal portion is formed of a material having an elastic modulus E ranging from between 7,000,000 psi to 10,000,000 psi.
